(19) United States
(12) Patent Application Publication
(10) Pub. No.: US 2024/0108896 A1
(43) Pub. Date: Apr. 4, 2024

(54) PERSONALIZED TRANSCRANIAL ALTERNATING CURRENT STIMULATION

(71) Applicant: StimScience Inc., Berkeley, CA (US)

(72) Inventor: Ram Gurumoorthy, Berkeley, CA (US)

(21) Appl. No.: 18/475,672

(22) Filed: Sep. 27, 2023

Related U.S. Application Data
(60) Provisional application No. 63/377,935, filed on Sep. 30, 2022.

(51) Int. Cl. A61N 1/36; A61N 1/04

(52) U.S. Cl. CPC A61N 1/36031; A61N 1/0456

(57) ABSTRACT

A method includes receiving, by at least one processor, an electroencephalogram (EEG) signal from a user's brain, identifying, with the at least one processor, two peak (EEG) frequencies in a 4-6 Hz band and a 9-11 Hz band of the received signal, generating, with the at least one processor, an alternating current electrical stimulation waveform by combining sinusoids at the two identified peak EEG frequencies, and applying, with at least one electrode, the alternating current electrical stimulation waveform to the user's brain.

### CROSS-REFERENCE TO RELATED APPLICATIONS

This application claims priority to and incorporates by references U.S. Provisional Patent Application No. 63/377,935 filed Sep. 30, 2022.

### TECHNICAL FIELD

This disclosure relates to transcranial alternating current stimulation (tACS) and more particularly, but not exclusively, to personalized tACS based on an individual's peak electroencephalogram (EEG) frequencies.

### BACKGROUND

Insufficient sleep is a major health issue. Inadequate sleep is associated with an array of poor health outcomes, including cardiovascular disease, diabetes, obesity, certain forms of cancer, Alzheimer's disease, depression, anxiety, and suicidality. Given concerns with typical sedative hypnotic drugs for treating sleep difficulties, there is a compelling need for alternative interventions.

### BRIEF SUMMARY

In one aspect, a method, includes receiving, by at least one processor, an electroencephalogram (EEG) signal from a user's brain, identifying, with the at least one processor, two peak (EEG) frequencies in a 4-6 Hz band and a 9-11 Hz band of the received signal, generating, with the at least one processor, an alternating current electrical stimulation waveform by combining sinusoids at the two identified peak EEG frequencies, and applying, with at least one electrode, the alternating current electrical stimulation waveform to the user's brain.

In one aspect, a non-transitory computer-readable storage medium includes instructions that when executed by at least one processor, cause the at least one processor to receive an electroencephalogram (EEG) signal from a user's brain, identify two peak (EEG) frequencies in a 4-6 Hz band and a 9-11 Hz band of the received signal, generate an alternating current electrical stimulation waveform by combining sinusoids at the two identified peak EEG frequencies for application to the user's brain with at least one electrode.

In one aspect, a system, includes at least one electrode. The system also includes at least one processor. The system also includes a memory storing instructions that, when executed by the at least one processor, configure the at least one processor to receive an electroencephalogram (EEG) signal from a user's brain, identify two peak (EEG) frequencies in a 4-6 Hz band and a 9-11 Hz band of the received signal, generate an alternating current electrical stimulation waveform by combining sinusoids at the two identified peak EEG frequencies for application to the user's brain with the at least one electrode.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an example of a system for providing customized electrical stimulation to enhance sleep, configured in accordance with some examples.

FIG. 2A illustrates age distribution of participants.

FIG. 2B illustrates ISI distribution of participants.

FIG. 3A illustrates fixed frequency blended 5 Hz & 10 Hz Current (mA) signal.

FIG. 3B illustrates personalized frequency blended 5.7 Mhz & 9.6 Hz Current (mA) signal.

FIG. 4A illustrates sleep duration comparison across conditions.

FIG. 4B illustrates onset comparison across stimulations.

FIG. 5 illustrates sleep duration versus age regression.

FIG. 6 illustrates a routine in accordance with one example.

FIG. 7 illustrates a routine in accordance with one example.

FIG. 8 illustrates an example of a computer system in accordance with one example.

### DETAILED DESCRIPTION

Reference will now be made in detail to some specific examples of the invention including the best modes contemplated by the inventors for carrying out the invention. Examples of these specific embodiments are illustrated in the accompanying drawings. While the present disclosure is described in conjunction with these specific examples, it will be understood that it is not intended to limit the invention to the described examples. On the contrary, it is intended to cover alternatives, modifications, and equivalents as may be included within the spirit and scope of the invention as defined by the appended claims. In addition, although many of the components and processes are described below in the singular for convenience, it will be appreciated by one of skill in the art that multiple components and repeated processes can also be used to practice the techniques of the present disclosure.

In the following description, numerous specific details are set forth in order to provide a thorough understanding of the present invention. Particular examples of the present disclosure may be implemented without some or all of these specific details. In other instances, well known process operations have not been described in detail in order not to unnecessarily obscure the present disclosure.

As will be discussed in greater detail below, various examples disclosed herein provide the ability to obtain measurements and provide stimulation via intracranial modalities and/or with surface or scalp level modalities. In addition, various examples may include other measurement modalities such as MEG, fMRI, NIRS, respiration, heart rate, electrooculography (EOG), temperature, body movements, oximetry, etc.

Furthermore, various examples disclosed herein provide enhancements in a user's ability to enter sleep and advance through stages of sleep. Examples disclosed herein provide a user with customized stimulation that is configured to help the user enter sleep and/or sleep longer.

Additional details will be discussed in greater detail below with reference to components of systems disclosed herein as well as stimulation.

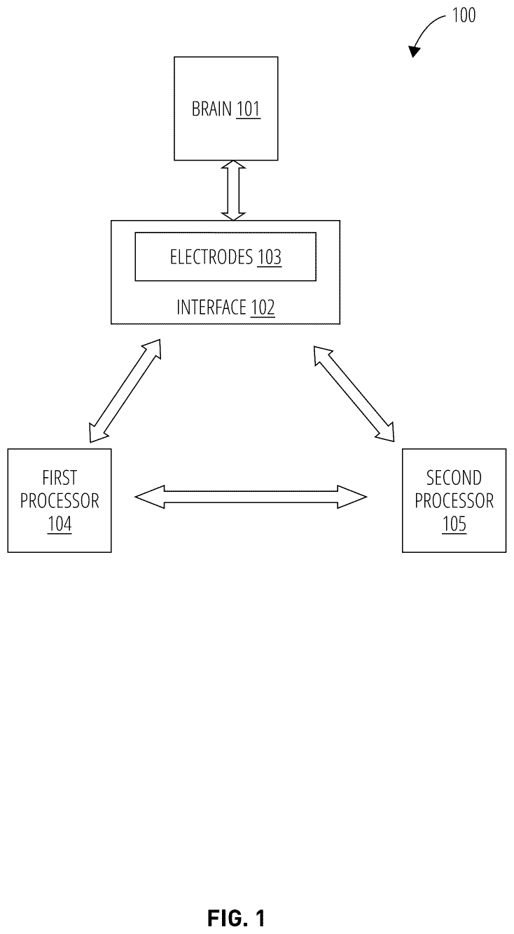

FIG. 1 illustrates an example of a system for providing customized electrical stimulation to enhance sleep, configured in accordance with some examples. In some embodiments, system 100 includes an interface, such as interface 102. In various embodiments, interface 102 is a brain interface that is configured to be coupled with a brain, such as brain 101. As will be discussed in greater detail below, such coupling may provide bidirectional communication, or may be used for various sensing modalities. In some embodiments, interface 101 includes various electrodes, such as electrodes 103, which may be included in an electrode array. Such electrodes may be included in a scalp potential electroencephalogram (EEG) array, may be deep brain stimulation (DBS) electrodes such as electrodes used with intracranial electroencephalography, and/or may be an epidural grid of electrodes. In other examples, interface 102 may include optogenetics mechanisms for monitoring various neuronal processes (central or peripheral). In other examples, interface 102 may include MEG, fMRI, NIRS, respiration, heart rate, electrooculography (EOG), temperature, body movements, oximetry devices. Mechanisms may be used to make various measurements and acquire measurement signals corresponding to neural activity. As used herein, neural activity may refer to spiking or non-spiking activity/potentiation.

In various examples, such measured signals may be electrical signals derived based on neural activity that may occur in cortical tissue of a brain. Such measurements may be acquired and represented in a time domain and/or frequency domain. In this way, neural activity may be monitored and measured over one or more temporal windows, and such measurements may be stored and utilized by system 100. In various embodiments, such neural activity may be observed for particular regions of cortical tissue determined, at least in part, based on a configuration of interface 102. In one example, this may be determined based on a configuration and location of electrodes included in interface 102 and coupled with the brain 101.

According to some embodiments, one or more components of interface 102 are configured to provide stimuli to the brain coupled with interface 102. For example, one or more electrodes 103 included in interface 102 may be configured to provide electrical stimuli to cortical tissue of the brain. As discussed above, such electrodes 103 may be implemented utilizing one or more of various modalities which may be placed on a user's scalp, or implanted in the user's brain.

As will be discussed in greater detail below, such actuation and stimuli provided by interface 102 may be of many different modalities. For example, stimuli may be aural, visual, and/or tactile as well as being electrical and/or magnetic, or any suitable combination of these. Accordingly, interface 102 may further include additional components, such as speakers, headphones, bone-conducting speakers, lights, display screens, and mechanical actuators that are configured to provide one or more of aural, visual, and/or tactile stimuli to a user. In this way, any suitable combination of different modalities may be used. For example, a combination of electrical and aural stimuli may be provided via interface 102. Further still, interface 102 may include different portions corresponding to signal acquisition and stimuli administration. For example, a first portion of interface 102 may include electrodes configured to measure neural activity, while a second portion of interface 102 includes speakers configured to generate aural stimuli.

In some embodiments, interface 102 further includes one or more dedicated processors and an associated memory configured to obtain and store the measurements acquired at interface 102. In this way, such measurements may be stored and made available to other system components which may be communicatively coupled with interface 102.

System 100 further includes at least one processor, e.g., a first processor 104 and a second processor 105, which are configured to received brain measurements from the interface 102 and, based on the received measurements, command the interface 102 to electrically stimulate the brain 101 with the electrodes 103 as will be discussed further below.

In one example, a preliminary session can be conducted to collect EEG data from a user to identify, on an individual basis, the power peaks within the alpha and theta bands of the brain 101. These data can be obtained during a 15-min daytime session, with the participant in a relaxed, eyes-closed state, before any of the pre-sleep stimulation sessions. The interface 102 may have 2 channels of EEG with electrodes at frontal-lobe sites of Fp1 and Fp2 using a bipotential reference electrode at Fpz.

The EEG signal is then band pass filtered with cutoff frequencies at 0.3 and 45 Hz. At least one process can then calculate the power spectral density (PSD) using the Welch method on the filtered data and the Fooof algorithm is used to determine frequency peaks after removing the aperiodic component of the spectrum. A k-means algorithm is used to calculate all peaks between 3 and 12 Hz. Two peaks are identified from these peaks, the first selected as the peak closest to 5 Hz within 4-6 Hz band and second selected as the peak closest to 10 Hz within 9-11 Hz band. The stimulation waveform for the personalized condition is then created by combining the sinusoids at the identified two peak frequencies. The amplitude of both sinusoids are set to 0.6 mA (peak-to-peak). At least one processor then caused the electrodes 103 to issue the stimulation before the participant went to sleep. Note that the two sinusoids in the Personalized stimulation protocol were started in phase, but they were not harmonics.

System 100 and its respective components may be implemented in a variety of contexts. For example, system 100 may be implemented in a clinical setting that may include an examination room, an operating room, or an emergency room. Moreover, system 100 may be implemented in a user's home thus providing in-home monitoring, diagnostic, and treatment. Furthermore, portions of system 100 may be implemented in a first location while other portions are implemented in a second location. For example, interface 102 may be located at a user's home, while the first processor 104 and/or the second processor 105 are implemented remotely, as may be the case when implemented at a hospital or when using cloud computing.

Furthermore, system 100 may be implemented across multiple users. For example, system 100 may include multiple interfaces that are coupled with multiple brains. In this way, measurements may be made from multiple users, and stimuli may be provided to multiple users. In one example, measurements from a first user may be used to generate and provide stimuli to a second user. In this way, synchronization of at least part of a brain state may be implemented across multiple users.

### Materials and Methods

### Overview

Participants were tested over multiple sessions in their homes, self-administering the stimulation (Fixed tACS, Personalized tACS, or control) using a custom-built stimulator comprising a headband that contained two electrodes 103 positioned over the frontal lobes. The participants used a customized phone app (e.g., using the first processor 104) that randomly determined the stimulation mode for each of the alternating weeks. Sleep onset and duration were measured with a wearable tracker (Fitbit watch). The participants put the headband and tracker on when they were ready to go to sleep and used the phone app to start the stimulation.

### Participants and Protocols

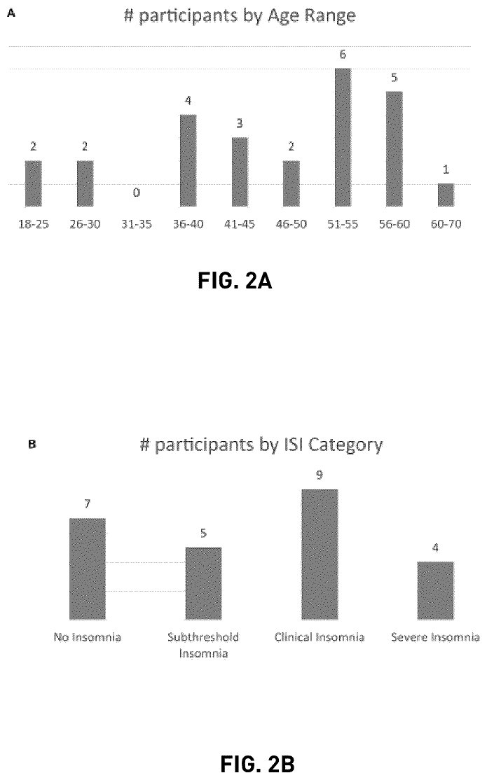

A total of 25 participants were tested in a repeated-measures, cross-over design (mean age: 46.3, with the age ranging between 19 and 60, 10 male and 15 female; FIG. 2A). Participants completed the Insomnia Severity Index questionnaire prior to their first session using an online form FIG. 2B. There was substantive representation of the ISI categories across the participants, with 28% categorized with no insomnia, 20% with subthreshold insomnia, 36% with clinical insomnia, and 16% with severe insomnia.

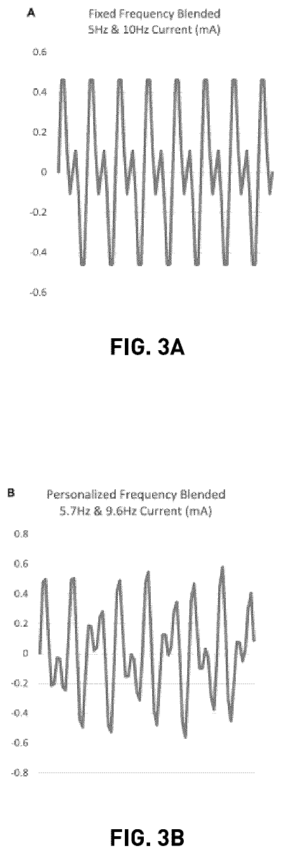

For the Fixed tACS condition, a stimulation waveform composed of two sinusoids was created, one at 10 Hz (targeting the alpha band) and one at 5 Hz (targeting the theta band). The two components were started in phase and summed to a maximal possible amplitude with every other cycle of the 10 Hz signal (FIG. 3A). These two frequency bands were targeted given their association with sleep onset, with alpha band activity (8-13 Hz) linked to stage I sleep and theta band activity (4-8 Hz) linked to the transition to stage II sleep. The amplitude of both sinusoids was set at 0.6 mA intensity (peak-to-peak) (FIG. 3B). For the Personalized tACS condition, a preliminary session was conducted to collect EEG data from the participants to identify, on an individual basis, the power peaks within the alpha and theta bands. These data were obtained during a 15-min daytime session, with the participant in a relaxed, eyesclosed state, before any of the pre-sleep stimulation sessions. The custom stimulator device had 2 channels of EEG with electrodes at frontal-lobe sites of Fp1 and Fp2 using a bipotential reference electrode at Fpz. The EEG signal was band pass filtered with cutoff frequencies at 0.3 and 45 Hz. The power spectral density (PSD) was calculated using the Welch method on the filtered data and the Fooof algorithm was used to determine frequency peaks after removing the aperiodic component of the spectrum. A k-means algorithm was used to calculate all peaks between 3 and 12 Hz. Two peaks were identified from these peaks, the first selected as the peak closest to 5 Hz within 4-6 Hz band and second selected as the peak closest to 10 Hz within 9-11 Hz band. The stimulation waveform for the personalized condition was created by combining the sinusoids at the identified two peak frequencies. The amplitude of both sinusoids was set to 0.6 mA (peak-to-peak). The two sinusoids in the Personalized stimulation protocol were started in phase, but they were not harmonics. FIG. 3A and FIG. 3B show the two blended signals (fixed frequency and a sample personalized frequency). Each participant used the device over a micro-longitudinal 2-week intervention period. For one of the weeks, the app was randomly set to deliver Fixed stimulation for 15 min. For the other week, the app was set to deliver Personalized stimulation for 15 min. Participants were asked to use the headset “as often as convenient,” with the recognition that they were unlikely to use the device on each night. Days in which the participants did not wear the headset (no stimulation) or when they failed to use the app to administer stimulation served as a Control condition (with data available only if they were wearing the tracker device). On average, Fixed stimulation was administered on 5 days (range: 3-6), Personalized on 4 days (range: 3-6), and 3 days of Control data was obtained (range: 1-6).

### Data Analysis

Sleep tracking data was obtained using a Fitbit tracker, the output of which provided sleep/wake durations for the participant through the night. Sleep stage data from the device were not analyzed since the classifier accuracy of different sleep stages for the Fitbit tracker are not sufficiently robust. On days with Fixed or Personalized tACS, sleep onset was defined as the interval between the end of stimulation, with the customized phone app notifying the end of stimulation, and the start of the first sleep epoch determined by the tracker data. As stimulation sensation was noticeable for the participant, we used the end of stimulation as the event from which to begin measuring sleep onset. Given that there was no stimulation in the Control condition, sleep onset data (as defined to be measured from the end of stimulation) was not recordable for this condition.

Data outliers were defined based on two pre-hoc criteria: (1) If the sleep start time for a session was beyond ±1.5 h of their sleep start time distribution inter-quartile-range, or (2) the participant's sleep duration fell beyond ±1.5 h of their sleep duration distribution inter-quartile-range. On average, 0.3 sessions (range: 0-2) were excluded and the distribution was similar across the conditions. From the remaining data, mean sleep onset and sleep duration scores for each participant were calculated in each condition. Note that the Control data were collected across the 2-week study period.

### Results

Personalized Vs. Fixed tACS Stimulation

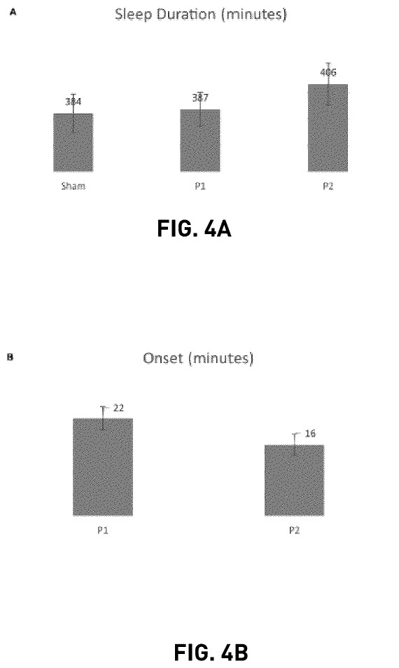

Personalized tACS stimulation increased sleep duration by 22 min compared to the Control condition (p=0.04) and 19 min compared to Fixed stimulation (p=0.03; see FIG. 4A). Fixed stimulation increased sleep duration by 3 min compared to Control condition, but this difference was not significant (mean: 3 min; p=0.75). Personalized stimulation resulted in a faster sleep onset by 6 min compared to the Fixed stimulation (28% improvement, p=0.02, refer to FIG. 4B and Tables 1, 2). Using the demographic information collected concerning age and sleep hygiene, two secondary analyses were conducted on sleep duration (Table 3). For the age analysis, we divided the participants into two groups based on age: ≤50 years old with n=13, >50 years old with n=12.

TABLE 1
Personalized (P2) - Fixed (P1)

| | Sleep Duration | Onset |
|---|---|---|
| Mean | 19 | −6 |
| P-value | 0.03 | 0.02 |

Sleep duration and onset performance

TABLE 2
Sleep duration compared to sham

| | Fixed (P1) | Personalized (P2) |
|---|---|---|
| Mean | 3 | 22 |
| P-value | 0.75 | 0.04 |

Sleep duration compared to sham

TABLE 3
Personalized (P2) - Fixed (P1)

| | Young | Old | | |
|---|---|---|---|---|
| | (≤50 years old | (>50 years old) | ISI 1 or 2 | ISI 3 or 4 |
| Mean | 27 | 10 | 4 | 33 |
| P-value | 0.02 | 0.45 | 0.67 | 0.02 |

Sleep duration by age and ISI

### Age Analysis

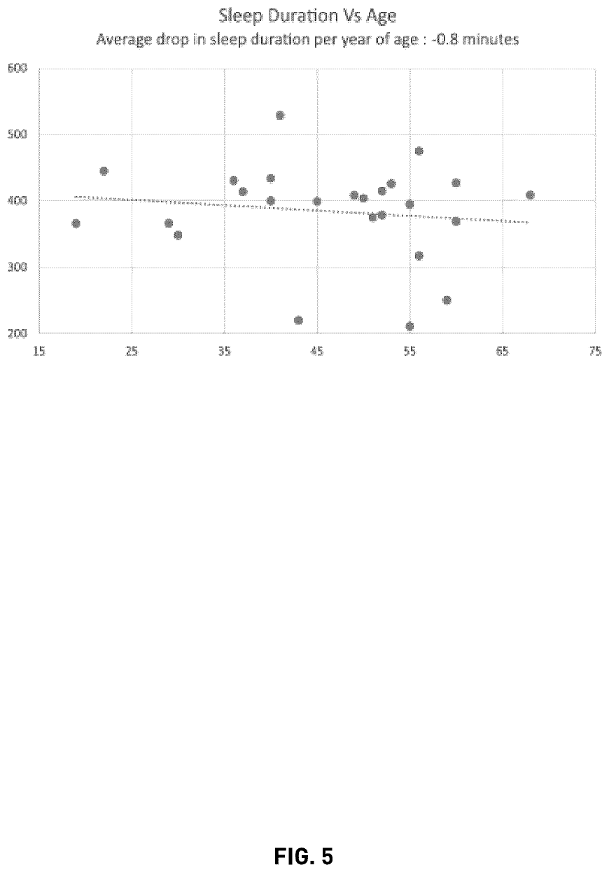

First, to confirm the classic age-related decrease in sleep as a validation of the cohort and its normative sleep, there was a significant correlation between age and sleep duration (data from Control condition, r=−0.19, p<0.001), with an average −0.8-min decrease in sleep duration with every year increase in age (FIG. 5), consistent with published norms.

Segmenting the cohort based on age, and first focusing on the younger cohort (≤50 years old), Personalized tACS stimulation resulted in a 27-min increase in sleep duration, relative to Fixed tACS stimulation (p=0.02), and a 29-min increase in sleep duration compared to the Control condition (p=0.02). In the older cohort (>50 years old), Personalized tACS stimulation elicited a non-significant 10-min increase in sleep duration, relative to Fixed tACS stimulation (p=0.4: ns) and an also non-significant 14-min increase relative to Control condition (p=0.45: ns).

Poor- vs. Good-sleeper Analyses: Based on sleep hygiene, the cohort was further segmented post hoc into two groups: a normative sleep group (those with no-insomnia and subclinical threshold insomnia ISI categorization, n=12), and a poor sleep group (those with clinical insomnia and severe insomnia ISI categorization, n=13). For the poor sleep group, Personalized tACS stimulation improved sleep duration by 33 min compared to Fixed tACS stimulation (p=0.02), and 30 min sleep duration compared to Control condition (p<0.1: ns). For the normative sleep group, Personalized tACS stimulation increased sleep duration by 4 min relative to Fixed tACS stimulation (p=0.67; ns) and increased 13 min compared to Control condition (p=0.2; ns).

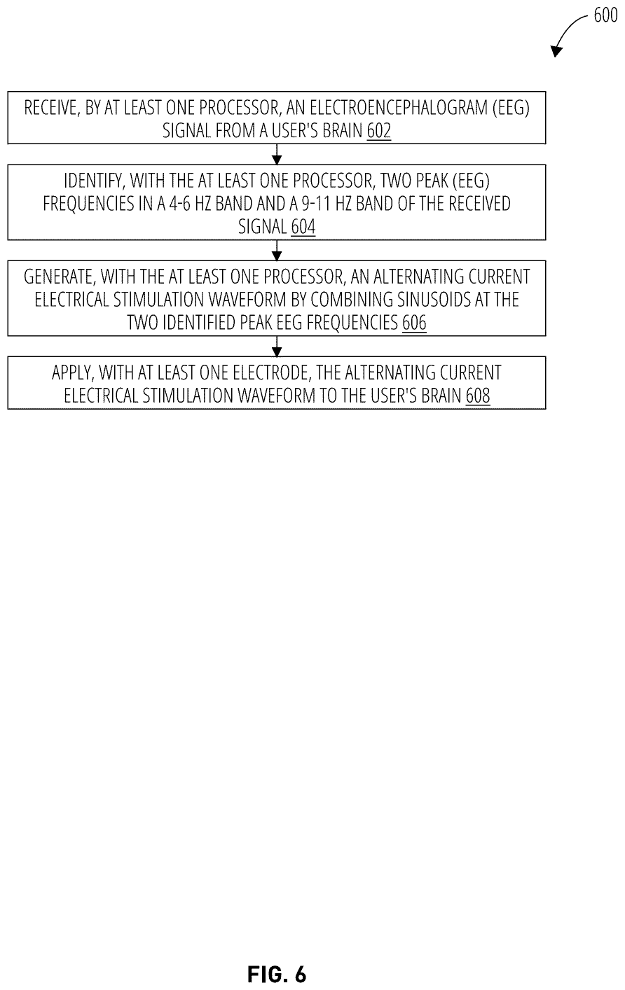

FIG. 6 illustrates an example routine 600. Although the example routine 600 depicts a particular sequence of operations, the sequence may be altered without departing from the scope of the present disclosure. For example, some of the operations depicted may be performed in parallel or in a different sequence that does not materially affect the function of the routine 600. In other examples, different components of an example device or system that implements the routine 600 may perform functions at substantially the same time or in a specific sequence.

In block 602, at least one processor receives an electroencephalogram (EEG) signal from a user's brain. In block 604, the at least one processor identifies two peak (EEG) frequencies in a 4-6 Hz band and a 9-11 Hz band of the received signal. In block 606, the at least one processor generates an alternating current electrical stimulation waveform by combining sinusoids at the two identified peak EEG frequencies. In block 608, at least one electrode applies the alternating current electrical stimulation waveform to the user's brain.

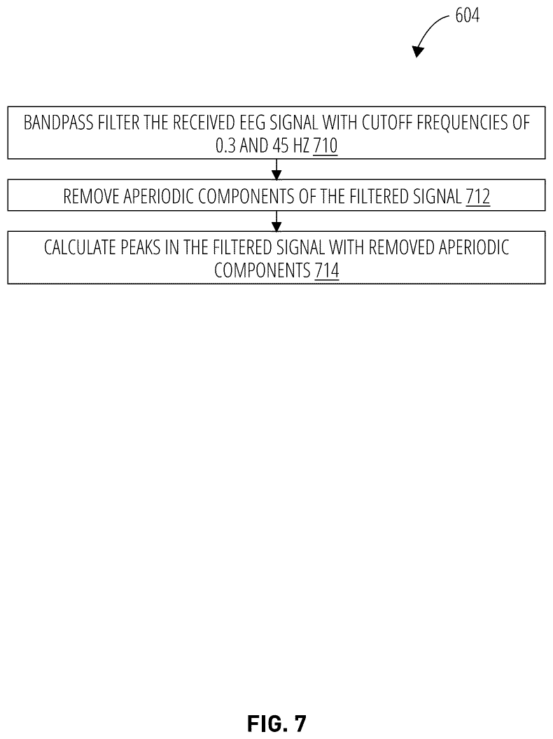

FIG. 7 illustrates an example of the operations of the block 604. In block 710, a filter bandpass filters the received EEG signal with cutoff frequencies of 0.3 and 45 Hz. In block 712 comprises removes aperiodic components of the filtered signal. In block 714, the at least one processor calculates peaks in the filtered signal with removed aperiodic components.

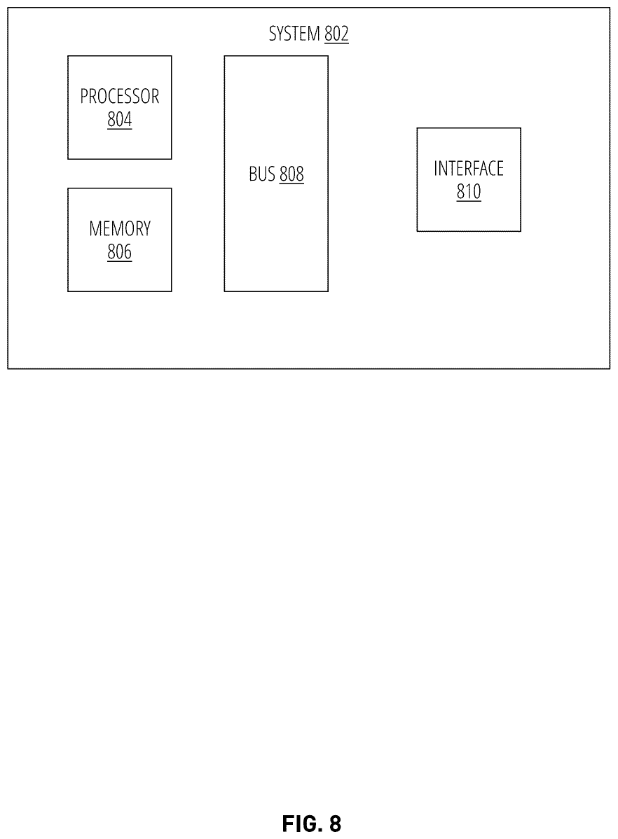

FIG. 8 illustrates an example of a computer 802 that can be used with various embodiments. For instance, the computer system 802 can be used to implement the first processor 104 and/or the second processor 105 according to various examples described above. In addition, the computer system 802 shown can represent a computing system on a mobile device or on a computer or laptop, etc. According to particular examples, a system 802 suitable for implementing particular examples includes a processor 804, a memory 806, an interface 810, and a bus 808 (e.g., a PCI bus). The interface 810 may include separate input and output interfaces, or may be a unified interface supporting both operations. When acting under the control of appropriate software or firmware, the processor 804 is responsible for tasks such as closed loop control. Various specially configured devices can also be used in place of an 804 or in addition to processor 804. The complete implementation can also be done in custom hardware. The interface 810 is typically configured to send and receive data packets or data segments over a network. Particular examples of interfaces the device supports include Ethernet interfaces, frame 10 relay interfaces, cable interfaces, DSL interfaces, token ring interfaces, and the like.

In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control such communications intensive tasks as packet switching, media control and management.

According to particular example embodiments, the system 802 uses memory 806 to store data and program instructions and maintain a local side cache. The program instructions may control the operation of an operating system and/or one or more applications, for example. The memory or memories may also be configured to store received metadata and batch requested metadata.

Because such information and program instructions may be employed to implement the systems/methods described herein, the examples relate to tangible, machine readable media that include program instructions, state information, etc. for performing various operations described herein. Examples of machine-readable media include hard disks, floppy disks, magnetic tape, optical media such as CD-ROM disks and DVDs; magneto-optical media such as optical disks, and hardware devices that are specially configured to store and perform program instructions, such as read-only memory devices (ROM) and programmable read-only memory devices (PROMs). Examples of program instructions include both machine code, such as produced by a compiler, and files containing higher level code that may be executed by the computer using an interpreter.

Comparing the two active stimulation conditions, the results show that the Personalized tACS stimulation improves sleep duration and sleep onset relative to Fixed tACS stimulation (19 more minutes of sleep and 6 min earlier onset of sleep).

An age-related analysis validated the known sleep norms of sleep duration decreasing with age (decrease of 0.8 min per year observed with the Control condition). Previous studies have shown that night-to-night and inter-participant variability in sleep quality increases in older cohorts. Given these norms of aging and sleep quality deterioration, we performed a post hoc analysis of the impact of Personalized tACS stimulation on sleep duration relative to Control condition, for a younger cohort (≤50 years old) and an older cohort (>50 years old). We observed a robust 29-min increase in sleep duration for the younger cohort. For the older cohort, there was a non-significant sleep duration increase of 14-min, providing a potential for using the Personalized tACS stimulation to improve sleep quality with age.

In addition to the age factor, insomnia symptomatology was further analyzed. This was motivated by the factor that current sedative hypnotics have non-trivial side effects, have challenging aspects of long-term efficacy, may fail to implement normative sleep physiology, and the recent American College of Physicians recommendation that they should no longer be a first line treatment approach for those with sleeping difficulties.

Post hoc analysis demonstrated that the poor sleep subgroup (defined as having significant insomnia categorization using the ISI) showed a larger boost in sleep duration (33 min increase with an effect size of 0.55) from personalized tACS stimulation compared to the Fixed tACS protocol. As a point of contrast, the typical prescription sleep medication, zolpidem (brand name, Ambien), has been shown to increase total sleep time by 35.5 min relative to placebo, and a more recent medication, suvorexant, has a reported increase in total sleep time of 28 min. As such, the current results show that non-invasive, personalized stimulation is a viable alternative intervention for insomnia.

Finally, the current study administered the tACS stimulation for 15-min pre-sleep and involved no additional stimulation during sleep. Further examples include. first, resting state EEG could be obtained prior to each night to account for within-subject fluctuations across days in peak frequency. Second, EEG could be monitored during sleep to allow for additional personalized stimulation during the night.

While the present disclosure has been particularly shown and described with reference to specific examples thereof, it will be understood by those skilled in the art that changes in the form and details of the disclosed examples may be made without departing from the spirit or scope of the invention. Specifically, there are many alternative ways of implementing the processes, systems, and apparatuses described. It is therefore intended that the invention be interpreted to include all variations and equivalents that fall within the true spirit and scope of the present invention. Moreover, although particular features have been described as part of each example, any combination of these features or additions of other features are intended to be included within the scope of this disclosure. Accordingly, the examples described herein are to be considered as illustrative and not restrictive.

What is claimed is:

1. A method, comprising:
receiving, by at least one processor, an electroencephalogram (EEG) signal from a user's brain;
identifying, with the at least one processor, two peak (EEG) frequencies in a 4-6 Hz band and a 9-11 Hz band of the received signal;
generating, with the at least one processor, an alternating current electrical stimulation waveform by combining sinusoids at the two identified peak EEG frequencies; and
applying, with at least one electrode, the alternating current electrical stimulation waveform to the user's brain.

2. The method of claim 1, wherein the identifying comprises:
bandpass filtering the received EEG signal with cutoff frequencies of 0.3 and 45 Hz;
removing aperiodic components of the filtered signal; and
calculating peaks in the filtered signal with removed aperiodic components.

3. The method of claim 1, wherein the identifying identifies a first peak closest to 5 Hz within the 4-6 Hz band and a second peak closest to 10 Hz with the 9-11 Hz band.

4. The method of claim 1, wherein amplitudes of the sinusoids is set to 0.6 mA.

5. The method of claim 1, wherein the applying is applied pre-sleep.

6. The method of claim 5, wherein the EEG signal is measured during daytime before the applying.

7. The method of claim 1, wherein the applying applies the waveform in phase.

8. The method of claim 1, wherein the sinusoids are not harmonics.

9. The method of claim 1, wherein the identifying includes calculating all peaks within 3 Hz and 12 Hz and identifying a first peak within the 4-6 Hz band and a second peak within the 9-11 Hz band.

10. A non-transitory computer-readable storage medium, the computer-readable storage medium including instructions that when executed by at least one processor, cause the at least one processor to:
receive an electroencephalogram (EEG) signal from a user's brain;
identify two peak (EEG) frequencies in a 4-6 Hz band and a 9-11 Hz band of the received signal; and
generate an alternating current electrical stimulation waveform by combining sinusoids at the two identified peak EEG frequencies for application to the user's brain with at least one electrode.

11. A system, comprising:
at least one electrode;
at least one processor; and
a memory storing instructions that, when executed by the at least one processor, configure the at least one processor to:
receive an electroencephalogram (EEG) signal from a user's brain;
identify two peak (EEG) frequencies in a 4-6 Hz band and a 9-11 Hz band of the received signal; and
generate an alternating current electrical stimulation waveform by combining sinusoids at the two identified peak EEG frequencies for application to the user's brain with the at least one electrode.

12. The system of claim 11, wherein the identifying comprises:
bandpass filter the received EEG signal with cutoff frequencies of 0.3 and 45 Hz;
remove aperiodic components of the filtered signal; and
calculate peaks in the filtered signal with removed aperiodic components.

13. The system of claim 11, wherein the identifying identifies a first peak closest to 5 Hz within the 4-6 Hz band and a second peak closest to 10 Hz with the 9-11 Hz band.

14. The system of claim 11, wherein amplitudes of the sinusoids is set to 0.6 mA.

15. The system of claim 11, wherein the application is applied pre-sleep.

16. The system of claim 15, wherein the EEG signal is measured during daytime before the applying.

17. The system of claim 11, wherein the application applies the waveform in phase.

18. The system of claim 11, wherein the sinusoids are not harmonics.

19. The system of claim 11, wherein the identifying includes calculate all peaks within 3 Hz and 12 Hz and identifying a first peak within the 4-6 Hz band and a second peak within the 9-11 Hz band.
